(19) United States
(12) Patent Application Publication
(10) Pub. No.: US 2003/0144630 A1
(43) Pub. Date: Jul. 31, 2003

(54) Safety syringe

(76) Inventors: Yu Kang Chang, Taiepi (TW); Ho-Song Cheng, Taiepi (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/055,977

(22) Filed: Jan. 28, 2002

(51) Int. Cl. A61M 5/32

(52) U.S. Cl. 604/198

(57) ABSTRACT

A safety syringe includes: a barrel, a plunger, a connecting member and a cover cap. Said barrel has a barrel tip with an injection passage at said barrel top. A plunger holds in said barrel. A connecting member covers over said barrel, embedded on said barrel tip. Said front part of connecting member forms at least a tab coupling with a extending a coupling shield in facing front to said tab coupling. Said connecting member has at least a coupling member and a seat bearing force from an elasticity element. At said connecting member top part has a locking wherein install a connected tip linked to said injection passage. A cover is capping said connecting member comprising a L-form socket which providing a socket path and a groove of said coupling member and tab coupling for coupling and fixing. A tuber front locates at the front part of said cover cap on which interior side bears force from elasticity element. Finally, as the above designed, such syringe device achieves the safety of after-use-syringe.

### FIELD OF THE INVENTION

A present invention relates to a safety syringe, in brief, which can be operated simply and safe.

### BACKGROUND OF THE INVENTION

For medical safe reason, the principle of syringe treatment is one-off used, then, used-syringe be sterilized and destroyed carefully to avoid repeated use or hurt people by uncontrolled discard. It is a highly risking period between syringe after used and before destroyed. However, even kinds of safety syringe developed, medical worker affect with fright of and to be aware that people hurt by used-syringe accident still happen due to not proper safety syringe designed or inconvenient syringe operated. Therefore, currently critical issue is to resolve the shortcoming of prior art, and to invent an operated simply, safe of new syringe designed and improve the security of operation people during syringe used to destroy.

### SUMMARY OF THE INVENTION

The primary object of present invention is to provide a safety syringe instrument device, which could highly promote safe, convenient and efficiency of syringe operation.

Another object of the present invention is to provide a safety syringe instrument device, by a simple operation procedure, to keep safe guard status during syringe used to destroy.

Yet another object of the present invention is to provide a safety syringe instrument device, in a safe guard status of used-syringe, with two-dimension protection and to ensure the safety of syringe-after-used.

The various objects and advantages of the present invention will be more readily understood from the following detailed description when read in conjunction with the appended drawings, in which:

### BRIEF DESCRIPTION OF THE DRAWINGS

FIG. 1 is an illustration diagram of the present invention.

FIG. 2 shows a schematic diagram for assembling of the present invention;

FIG. 3 shows a cross-sectional view of the present invention;

FIGS. 4 & 5 show schematic diagram for movement of the present invention;

FIG. 6 shows diagram for movement finished of the present invention;

FIGS. 7 & 8 show schematic diagram for protecting procedure movement of the present invention;

FIG. 9 shows a schematic diagram for assembling of another embodiment of present invention;

FIG. 10 shows a cross-sectional view of another embodiment of present invention;

FIGS. 11A & B show side view of another embodiment of present socket path invention;

### DETAILED DESCRIPTION OF THE PREFERRED EMBODIMENTS

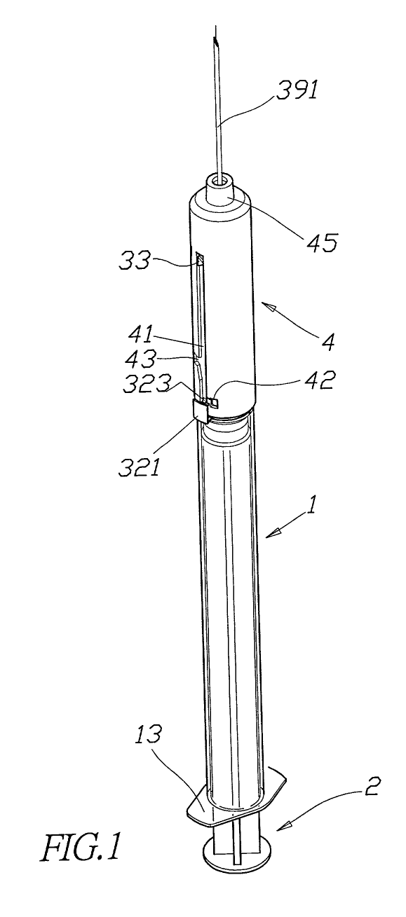

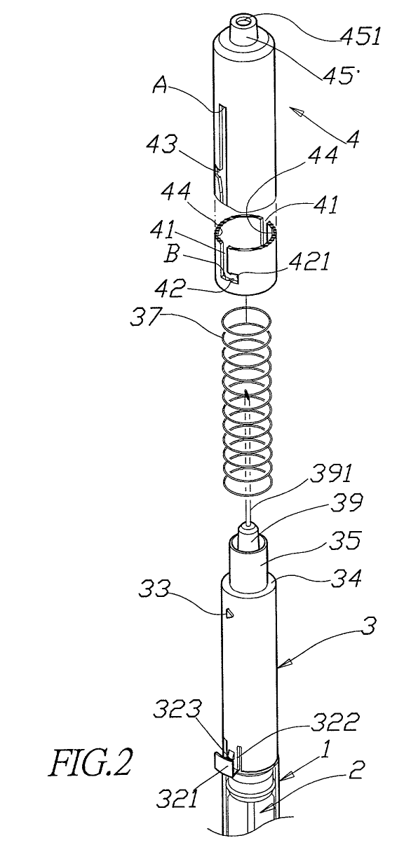

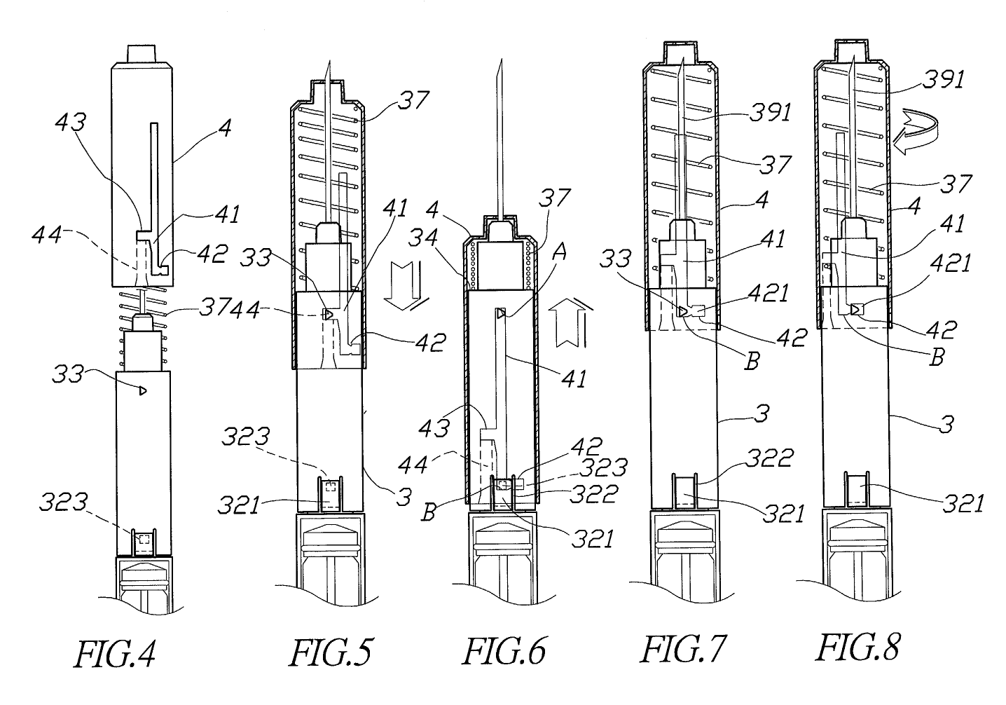

Referring FIGS. 1,2 & 3, a present invention includes a barrel 1, a plunger 2, a connecting member 3 and a cover cap 4. Said barrel 1 is a hollow cylinder with injection liquid filled inside, at a front arranged with a barrel tip 11, and a barrel joint 12. Said tip 11 contains an injection passage 111 for injection transmission purpose. Said joint 12, made to connect said connecting member 3, is an extended tube with a reduced diameter and associated with a space as joint space 123 to a retaining flange 122. Said joint 12 disposed to connect said injection passage 11 a linker channel 121, having a retaining flange 122. At rear end of said barrel 1 has a syringe handle flange 13 for easy handling. Said plunger 2, at inside of said barrel 1, provides an outward pressure to the inside liquid of said barrel 1 to pass through injection passage 111 which located in barrel tip. Said the connecting device of connected member 3 and said barrel joint 12 has two coaxial rings. The one inner connecting ring 31 near to core for completely inlaid to linker channel 121 associated with a fix flange 311 extended at said inner ring 31 end for either tightly covering said linker channel 121 or tight down in a thread connection. Besides, another outer connecting ring 32 arranged at least an extending snapping knob 321 which knob is projecting over said connecting member 3 and with side groove 322 enclosed at its two sides. Straight said snapping knob 321 top part forms a tab fitting coupling 323, and said tab-coupling 323 having another tab fitting as a coupling member 33, both of tab fittings are for coupling to the track of the socket path. The elements of said connecting member 3 top part contain a seat 34, a connecting joint 35 and a connected tip 36. Said seat 34 for bearing force from an elasticity element 37 located at periphery of said member 3, a connecting joint 35 and a connected tip 36 located at an inside of said connecting joint 35. A connected channel 38 is arranged between said connecting joint 35 and said connected tip 36 for tight fixing, supporting to a locking tip 39. Said connected channel 38 and said locking tip 39 could be connected in threading, capping or extruded. Additionally, in the interior side of said connected tip 36 has a central passage 361 linking to said injection passage 111 and to a needle cannula 391 for liquid outlet. Said cover cap 4 covering with said connecting member 3, an L-form socket path, contains at least a socket track 41 and a start-side-socket 42. Said socket track 41 links an end-side-socket 43 associated with a vertical direction and, on the direction to said barrel 1, has a guide socket 44 extending parallel with a socket track 41. Said guide socket 44 is formed at interior side of said cover cap 4 for guiding said coupling member 33 into said end-side-socket 43 and said socket track 41. Said start-side-socket 42, arrange a stopper cell 421 linking to said socket track 41 on another side of said end-side-socket 43. Said cover cap 4 top part comprises a tuber front 45 for settling said locking tip 39 in, containing an open mouth as a tuber front mouth 451 for said needle cannula 391 passing through, further, to bear force from elasticity element in said cap 4 interior top side. Shown in FIG. 4, before said cover cap 4 capping said connecting member 3, said guide socket 44 directed toward said coupling member 33 to lead said coupling member 33 filling and moving by said end-side-socket 43, guide socket 44, then into socket track 41. Further, shown as FIG. 5, to forward said coupling member 33 into the end track of said socket track 41, “A” mark. Shown as FIG. 6, said tab coupling 323, a tab-fit designed, connects to said cover cap 4 and fills into another end track of said socket track 41, “B” mark, at this moment, the space between seat 34 of said connecting member 3 and interior side of cover cap 4 is pressed hard by a elasticity element 37, additionally, the assembly of said cover cap 4 and said connecting member 3 is finished.

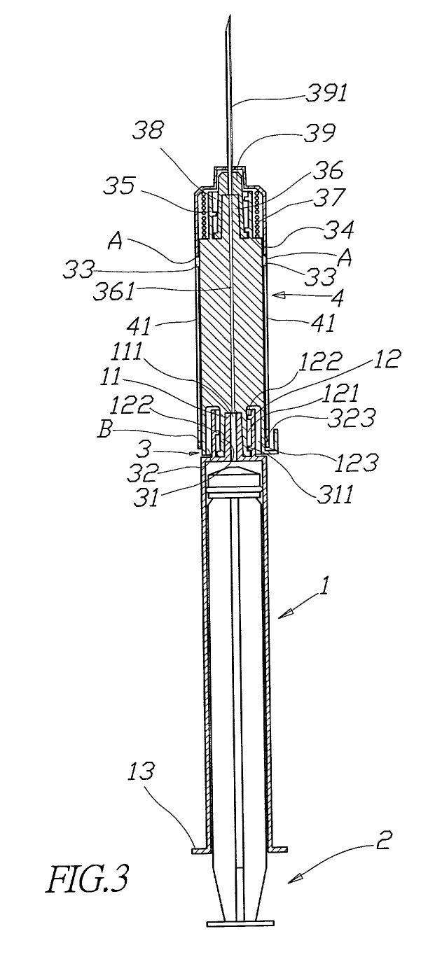

After-used-syringe is shown as FIG. 6 to FIG. 8, medical worker pressing said coupling shield 321. Since said coupling shield 321 has said side groove 322 around at two sides of them which make said coupling shield 321 easy to be pressed down and entering into said joint space 123 of said barrel 1. Further more, shown as FIG. 3, said tab coupling 323 will be out of coupling and connecting to said socket track 41, at meanwhile, said cover cap 4 forced by said elasticity element 37 to be spring outward and to seal up said needle cannula 391. In addition, said coupling member 33 moves by the track of said socket track 41,then, to be stock in the position “B” (mark as B). Moreover, to turn said cover cap 4 to guide said coupling member 33 to fill into said stopper cell 421, finally, to keep said cover cap 4 in safe guard status.

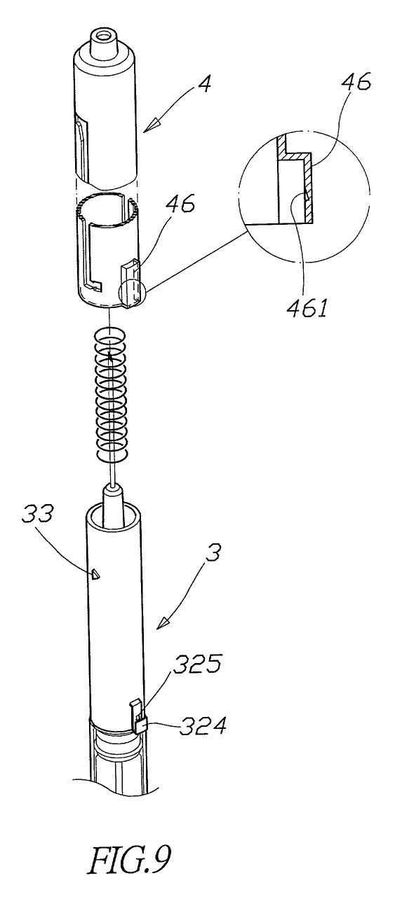

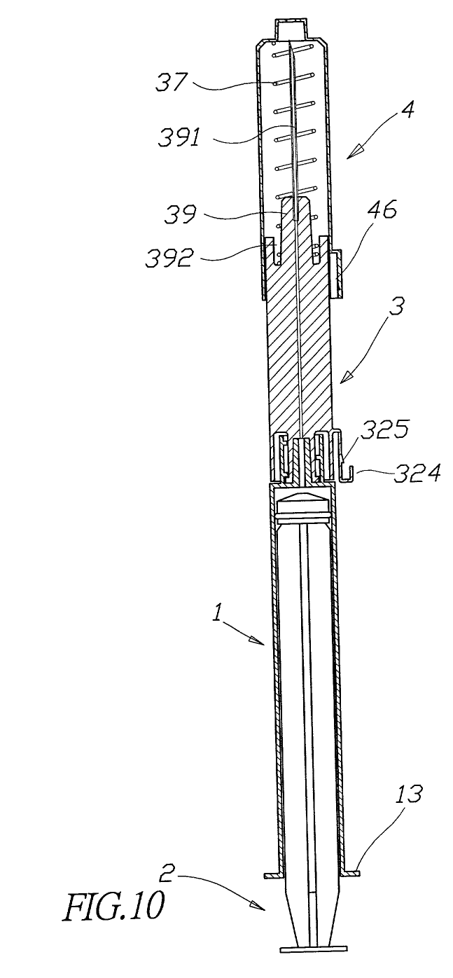

Another embodiment of present invention is shown as FIG. 9 and FIG. 10, said connecting member 3 has a press shield 324 which is a flexible cover, project over said connecting member 3 and having a tab coupling 325 for making a coupling connection relationship to said cover cap 4. Said cover cap 4, in facing front to said press shield 324, has an extending cover 46. At interior side of said cover 46 arranges a track 461 for couple matching to said tab coupling 325, in a safeguard status, further, said locking tip 39 in associated with said connecting member 3 being made extrusion. A needle cannula 391 places in said locking tip 39. A space as a linker channel 392 is surround said locking tip 39. Said locking tip 39 and the inside of said cover cap 4 top are bearing the force from two end of elasticity element 37.

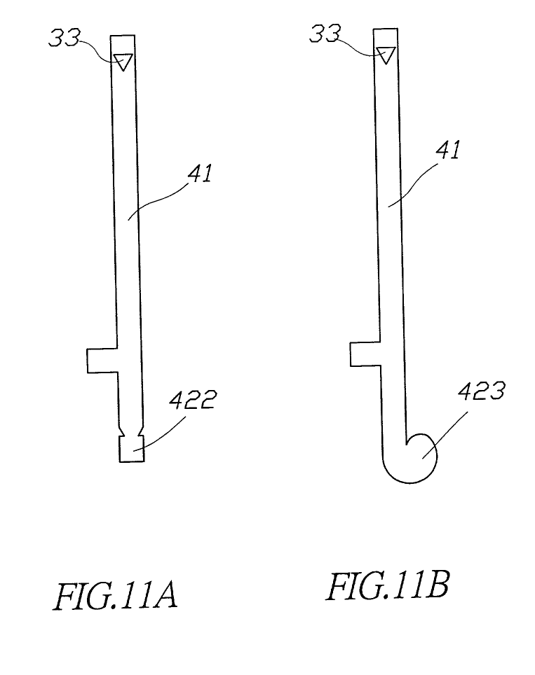

The other embodiment is shown in FIG. 11A and B, a stopper cell 422 straightly arranged at the end of said cover cap 4 or a stopper cell 423 arranged on the one side of said socket track 41 end, to achieve fixing forward and operating simply purpose.

According the above, a feature of the present invention is the provision a safe guard syringe by simple operation procedure, for security of medical worker from using syringe to destroy. Additionally, the present invention is more practically and safe, since there is a two-dimension protection designed to ensure the safety of used-syringe.

Many changes and modifications in the above-described embodiment of the invention can, of course, be carried out without departing from the scope thereof. Accordingly, to promote the process in science and the useful arts, the invention is disclosed and intends to be limited only by the scope of the appended claims.

I claim:

1. A safety syringe comprising,
a hollow cylinder barrel, having a barrel tip with an injection passage at a top thereof,
a plunger located inside of said barrel;
a connecting member mounted, embedded on said barrel tip, having at least a snapping knob at one side of said connecting member, wherein having a tab coupling in facing front to, arranged at least a coupling member and a seat which seat designs for bearing force of an elasticity element, at front part of said connecting member containing a locking tip having a central passage passed with a injection passage at a tip of said connecting member; and,
a cover cap, covering over said connecting member, comprising at least a socket track to provide a track for said coupling member coupling and said tab coupling grooving, at said connecting member front part arranging a front tab for containing said locking tip within a open mouth and in said cover cap interior side bearing forces from said elasticity element.

2. A safety syringe according to claim 1, wherein said barrel tip encloses a barrel joint which embedding closely on said connecting member.

3. A safety syringe according to claim 2, wherein said barrel joint outside is associated with a joint space.

4. A safety syringe according to claim 1, wherein said connecting member has a snapping knob mean an extending protrusible knob.

5. A safety syringe according to claim 4, wherein said snapping knob has a side groove surround.

6. A safety syringe according to claim 1, wherein said tab coupling means a tab of snapping in movable.

7. A safety syringe according to claim 1, wherein said connecting member has said locking top with a needle cannula embedded.

8. A safety syringe according to claim 7, wherein said locking tip and said connecting member could completely make extrusion.

9. A device as in claim 7, said locking tip is separated to said connecting member but connected to said needle cannula assembly.

10. A safety syringe according to claim 1, wherein said seat has a surround ring.

11. A safety syringe according to claim 1, wherein said cover cap has at least a track including a socket track and a start-side-socket.

12. A safety syringe according to claim 11, wherein said socket track links at least an end-side-socket associated with a vertical direction.

13. A safety syringe according to claim 12, wherein said end-side-socket links at least a guide socket, situated within said cover cap, extending parallel with said socket track and in the direction of said barrel.

14. A safety syringe according to claim 11, wherein said cover cap has at least a stopper cell for coupling said coupling member and said stopper cell located on another side of said start-side-socket.

15. A safety syringe according to claim 1, wherein said socket track at its end part extending has said stopper cell for coupling said coupling member.
